(19) United States
(12) Patent Application Publication
(10) Pub. No.: US 2026/0314966 A1
(43) Pub. Date: Oct. 15, 2026

(54) BIO-ELECTROCHEMICAL SENSORS

(71) Applicant: Tingo Medical Ltd., Modi'in (IL)

(72) Inventors: Guy Shinar, Ramat Gan (IL); Lior Shtram, Tel Aviv-Yaffo (IL); Yehonatan Ravenna, Luzit (IL); Idan Gal, Ramat HaSharon (IL); Marina Basovich, Pardes Hanna-Karkur (IL); Ofer Yodfat, Modi'in (IL)

(21) Appl. No.: 19/482,695

(22) PCT Filed: May 7, 2024
(86) PCT No.: PCT/IL2024/050436

§ 371 Date: Nov. 7, 2025

Related U.S. Application Data
(60) Provisional application No. 63/464,905, filed on May 8, 2023.

(51) Int. Cl. A61B 5/1486; A61B 5/145

(52) U.S. Cl. CPC A61B 5/14865; A61B 5/14532; A61B 5/14546

(57) ABSTRACT

In some embodiments of the present disclosure, a bio-electrochemical sensor for measuring the concentration of an analyte or substrate molecule (signal) is provided in which the electrical current (the response) is insensitive to oxygen, need not require a mediator or the sensing of a product molecular species, includes a low sensitivity to interferents and includes high operational stability in both the steady-state response magnitude and the response time.

### CROSS-REFERENCE TO RELATED APPLICATIONS

This application claims priority to U.S. Provisional Application No. 63/464,905, filed May 8, 2023, the disclosures of which are hereby incorporated by reference in its entirety for all purposes.

### FIELD OF THE DISCLOSURE

The field of the present disclosure is bio-electrochemical sensors. More specifically, the field of the present disclosure is bio-electrochemical sensors for implantation in a human subject.

### BACKGROUND OF THE DISCLOSURE

Electrochemical sensors transduce a chemical signal, such as the concentration of an analyte or a substrate of interest in a solution, to a response, such as an electrical current (amperometric), voltage (voltmetric) or charge (coulometric).

Bio-electrochemical sensors typically comprise a working electrode, an enzyme immobilized in a membrane layer (enzyme layer), and a layer configured to limit the flux of the substrate into the enzyme layer (substrate limiting layer). The substrate may transfer from the exterior of the sensor and into the substrate limiting layer, and from the substrate limiting layer into the enzyme layer. The enzyme may catalyze the conversion of the substrate into a product, with or without a co-substrate (co-factor) or a mediator.

Electrochemical sensors are characterized as first-generation whenever the product is a molecular species; as second generation whenever the product is the reduced form of a mediator; and as third generation (direct electron transfer) whenever the product is an electron. The working electrode may be sensitive to the product, and a current, a charge or a voltage difference may arise, whose magnitude, as compared to a reference electrode, a counter electrode or both, may be proportional to the analyte concentration (that is, the substrate concentration outside the sensor).

Third generation biosensors are conceptually advantageous over first-and second-generation biosensors because they provide a more direct measurement of analyte concentration than first- or second-generation sensors. They eliminate the need for an indirect measurement of the flux or concentration of a molecular product species, as in the first generation, which may degrade the sensor over time. They also eliminate the need for a mediator, as in the second generation, which adds complexity to the sensor chemistry. This simplicity may translate to higher manufacturing repeatability and/or lower cost.

Glucose sensors are an important subclass of biosensors. Desirable properties of glucose sensors include: operational stability—the maintenance of the steady-state response and the response time to a step change in the analyte (glucose) concentration, for a period of weeks; insensitivity to interferents, that is, molecular species present in the external environment other than the analyte of interest; simplicity, which is conducive to manufacturability and low cost.

Despite the conceptual attractiveness of third generation sensors, no third-generation, commercial, in-vivo glucose sensor exists, which has all the desirable properties described above. The present invention is directed at such a sensor.

### BRIEF DESCRIPTION OF THE FIGURES

FIG. 1 is a schematic of a biosensor according to some embodiments of the present disclosure, which includes a planar construction.

FIG. 2 is a schematic of a biosensor according to some embodiments of the present disclosure, which includes a cylindrical construction.

FIG. 3 includes three (3) chronoamperograms of a sensor according to some embodiments of the present disclosure, which include a CDH enzyme having a Michaelis constant of about 8 mM.

FIG. 4 includes three chronoamperograms of a sensor according to some embodiments of the present disclosure, which include a CDH enzyme having a Michaelis constant of about 80 mM.

### DESCRIPTION

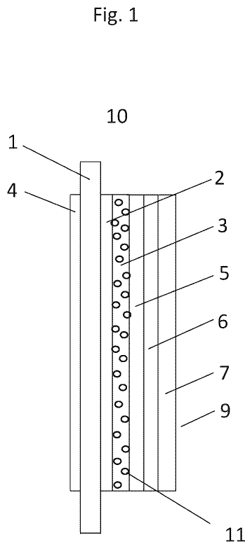

FIG. 1 illustrates a side-view of a bio-electrochemical sensor according to some embodiments of the present disclosure. As shown, sensor 10 can include a base 1, a working electrode 2, an enzyme layer 3, and a counter electrode, reference electrode, or counter/reference electrode 4. Optionally, sensor 10 may include one or more anti-interference layer 5, an analyte or glucose limiting layer 6, and a bio-interface layer 7. In operation, sensor 10 may be in contact with exterior 9, which may be the interstitial fluid of a human.

In some embodiments, sensor 10 may be a glucose sensor configured to sense the concentration of glucose in the exterior 9. In some embodiments, sensor 10 may be configured to sense the concentration of some other biomolecule of interest, such as, for example, lactate or a ketone, in the exterior 9. In some embodiments, two or more sensors 10 may be combined to measure the concentration of two or more analytes of interest.

In some embodiments, base 1 may be an insulator, such as a plastic or a ceramic. Examples of suitable plastics include, for example, polyimide, polyester, polycarbonate and polyamide.

In some embodiments, working electrode 2 may be made from a metal, such as, for example, platinum or gold. In some embodiments, working electrode 2 may be made from a non-metallic conductor such as carbon. In some embodiments, the working electrode may include carbon deposited on a metal. In some embodiments, counter electrode, reference electrode or counter/reference electrode 4 may be made from silver/silver chloride or gold. In some embodiments, electrodes 2 and 4 may be printed onto base 1 by, for example, screen printing.

In some embodiments, working electrode 2 may be operated at a voltage difference less than about 0.1V between the working electrode and the reference, or counter/reference electrode. In this way, catalysis by working electrode 2 of current-producing spurious electrochemical reactions, which include as reactants one or more interferent molecules present in environment 9, is limited. Sensor 10 may thus be more specific to its analyte of interest, and therefore more accurate than sensors operated at a voltage difference greater than or equal to 0.1V. In some embodiments, the voltage difference may be in the range of −0.2V to 0.2V In some embodiments, enzyme layer 3 may include a thickness of greater than 5 microns and includes an enzyme and a polymer. In some embodiments, the thickness of the enzyme layer 3 may be less than 5 microns. In some embodiments, the enzyme layer may also include a crosslinker. In some embodiments, the enzyme layer may also include conducting particles 11, which may be made from materials such as carbon black, carbon nanotubes or gold particles. In some embodiments, the enzyme layer may include a conducting polymer, such as polyethylenedioxythiophene (PEDOT), poly(3,4-ethylenedioxythiophene) polystyrene sulfonate (PEDOT:PSS), or polyaniline (PANI). In some embodiments, the enzyme layer may include a molecule configured to covalently connect the enzyme, the conducting particles, the polymer or the crosslinker to the working electrode, such as dithiobis(succinimidylhexanoate) (HEX). In some embodiments, the enzyme layer may include diethylaminoethyl (DEAE) and/or branched polyethylamine (BPEI).

In some embodiments, the enzyme may be immobilized in the enzyme layer. In some embodiments, the enzyme may have glucose, lactate or a ketone molecule as its substrate. In some embodiments, the enzyme may be configured to directly transfer electrons to the working electrode 2 and/or conducting particles 11 and/or the conducting polymer upon conversion of substrate to product (third generation enzyme). In some embodiments, the enzyme may be a genetically-modified cellobiose dehydrogenase (CDH), configured to have glucose as a substrate. In some embodiments, the enzyme may be a genetically-modified CDH or a genetically modified glucose dehydrogenase (GDH), configured to directly transfer electrons to the working electrode and/or conducting particles 11 and/or conducting polymer upon conversion of substrate to product.

In some embodiments, the enzyme may possess Michaelis Menten kinetics. In some embodiments, only the analyte of interest will be a substrate, and no co-substrate, such as oxygen, will participate in the chemical reaction catalyzed by the enzyme. In some embodiments, the Michaelis constant of the enzyme (that is, the substrate concentration in which half the enzyme molecules are bound to the substrate in solution) will be less than 30 mM. In some embodiments, the Michaelis constant of the enzyme will be less than 10 mM. Whenever glucose is the substrate, having the Michaelis constant less than 10 mM is counterintuitive because in solution it makes the enzyme's response non-linear within the physiological glucose range. In some embodiments, the enzyme will be a genetically modified CDH whose substrate is glucose, configured for direct electron transfer, and having a Michaelis constant that is less than about 10 mM.

One or more anti-interference layer 5 may be configured to diminish the diffusion of molecules such as ascorbic acid, uric acid and acetaminophen, which may be, for example, electro-oxidized at working electrode 2, and thus, can contribute a current that is independent of the analyte or substrate concentration in exterior 9. One or more anti-interference layer 5 may be constructed, for example, from cellulose acetate, Nafion™, Eastman AQ™, and polyallylamine, to prevent, for example, interference from acetaminophen, ascorbic acid and uric acid. One or more anti-interference layer 5 may be situated between working electrode 2 and enzyme layer 3, between enzyme layer 3 and some other layer, or both.

Substrate limiting layer 6 may be configured to diminish the flux of the substrate from exterior 9 into enzyme layer 3. The substrate limiting layer may reduce the flux of the substrate or analyte by a factor of 10-500, while allowing the free, or nearly free passage of a necessary cofactor, such as oxygen. In this way, the enzyme might be saturated by the cofactor under the range of expected operating conditions. If the enzyme is glucose oxidase (GOX), then oxygen can be used as a co-substrate to ultimately generate the product—hydrogen peroxide, To maintain the enzyme in the linear range for glucose, it is desirable that an oxygen concentration in enzyme layer 3 be much larger than the glucose concentration. This is achieved by the substrate (glucose in this case) limiting layer 6, which may diminish the glucose flux by a factor of 100 or more compared to the oxygen flux. Whenever the enzyme in enzyme layer 3 has the analyte as sole substrate, without requiring a co-substrate (in some embodiments), the substrate limiting layer 6 may be configured only to reduce the flux of the substrate or analyte, without care for whether the flux of other molecules, such as oxygen, do or do not have their flux reduced Whenever the enzyme does not require a co-factor, as, for example, in the case of CDH or GDH, the flux reduction factor afforded by substrate limiting layer 6 may be less than about 100, which, as compared to substrate limiting layers with larger reduction factors, is conducive to higher signal to noise ratio and better accuracy, while at the same time enhancing operational stability and endowing the sensor with linearity. Substrate limiting layer 6 may be made, for example, from polyurethanes, block copolymers of polyurethanes or silicones.

In some embodiments, bio-interface layer 7 may be configured to make sensor 20 biocompatible with the exterior 9, which may be, for example, subcutaneous tissue. Bio-interface layer 7 may be made, for example, from hydrogels, polyethylene oxide polymers, chitosans, and/or zwitter ions polymers.

In some embodiments, sensor 10 may include the following: (1) an insulator 1 such as polyimide; a working electrode 2 including, for example, gold or carbon; (3) a counter electrode made from, for example, gold; (4) a reference electrode made from, for example, silver/silver chloride, gold or platinum; (5) an enzyme layer 2 including a polymer matrix made, for example, from branched polyethyleneimine (BPEI), diethylaminoethyl (DEAE), or both, carbon particles sized about 0.3-0.5 microns, PEDOT or both, and oxygen-independent CDH enzyme genetically engineered for: (a) direct electron transfer, and (b) glucose as its sole substrate; (6) a glucose limiting layer 6 including, for example, a polyurethane. Glucose limiting layer 6 may be configured to reduce the glucose flux by a factor of 100 or less. The CDH enzyme may have a Michaelis constant of 10 mM or less.

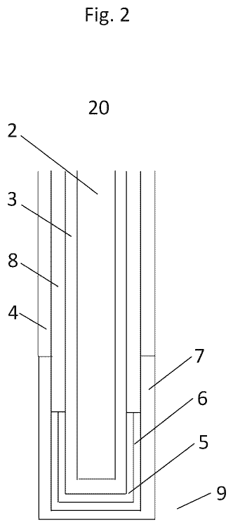

FIG. 2 illustrates a cross-section of a bio-electrochemical sensor 20 according to some embodiments of the present disclosure. Unlike sensor 10, which has planar geometry, sensor 20 has cylindrical geometry, Accordingly, sensor 20 may comprise a conducting wire 2 that also serves as a working electrode, an enzyme layer 3, an insulating layer 8 and a reference, a counter, or a counter/reference electrode 4. Working electrode 2, enzyme layer 3 and counter, reference, or counter/reference electrode 4 may be substantially similar in composition to the corresponding structures described above for sensor 10. Insulating layer 8 may be made from a plastic. The various layers that sensor 20 comprises may be deposited on wire 2 by, for example, dip coating. In addition, sensor 20 may also comprise one or more anti-interference layer 5, a glucose limiting layer 6, and a bio-interface layer 7. Layers 5, 6 and 7 of sensor 20 may be substantially similar to sensor 10.

### EXAMPLES

Example 1: Consider a sensor 10 according to an embodiment of the present invention, which is characterized by the following:
Substrate molecule: glucose
Base: polyimide
Working electrode: gold
Operating voltage difference between the working and counter/reference electrode: 0.1V
Enzyme layer:
   Enzyme: CDH, genetically engineered for direct electron transfer and having a Michaelis constant of about 8 mM for glucose (T3 from DirectSens, Vienna, Austria)
   Polymer matrix: BPEI 750 KDa (Sigma Aldrich, St. Louis, Missouri, USA)
   Carbon particles: Mesoporous carbon black <0.5 μm (Sigma Aldrich, St. Louis, Missouri, USA)
Glucose limiting layer: a blend polyurethanes (G25 and Carbosil, DSM, Geleen, The Netherlands)
Bio-interface layer: Lipidure CM-5206 (NOF Europe GMBH, Frankfurt am Main, Germany)

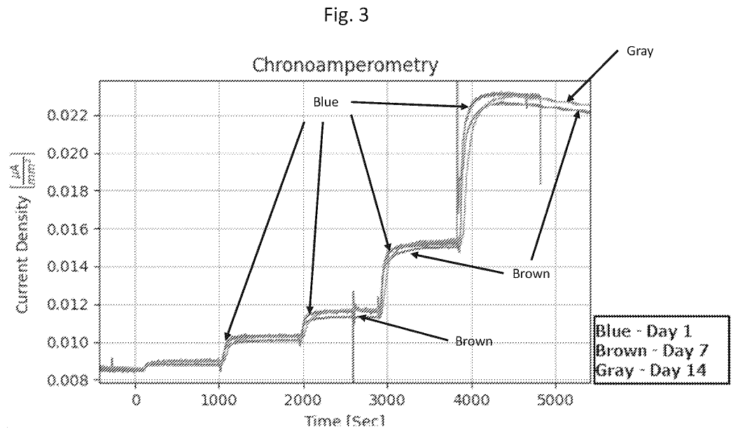

FIG. 3 shows chronoamperograms of a sensor 10 as above from days 1, 7 and 14. The chronoamperograms were generated by placing the sensor in a PBS solution and making step increases in the glucose concentration (1, 3, 5, 10, and 20 mM). (In between measurement days the sensor was kept activated at 5 mM glucose PBS solution.) The sensors were operated at 0.1V voltage difference between the working and counter/reference electrodes. The chronoamperograms were very similar between days 1, 7 and 14, demonstrating excellent operational stability in both the steady-state magnitude of the response to the step increases in glucose concentration, as well as the response time (always less than 300 s). Thus, sensor 10 of the present example has the following desirable properties: it is insensitive to the ambient oxygen concentration, because the CDH enzyme it includes is insensitive to oxygen; it operates a very low voltage difference between the working and the counter/reference electrodes, thereby making it less sensitive to interferents; it operates by direct electron transfer, thereby avoiding the production of corrosive molecular species (such as hydrogen peroxide in GOX-based first-generation sensors) or use of toxic mediators (such as osmium in second-generation GOX-based sensors); it has excellent operational stability, in both the steady-state response magnitude and the response time.

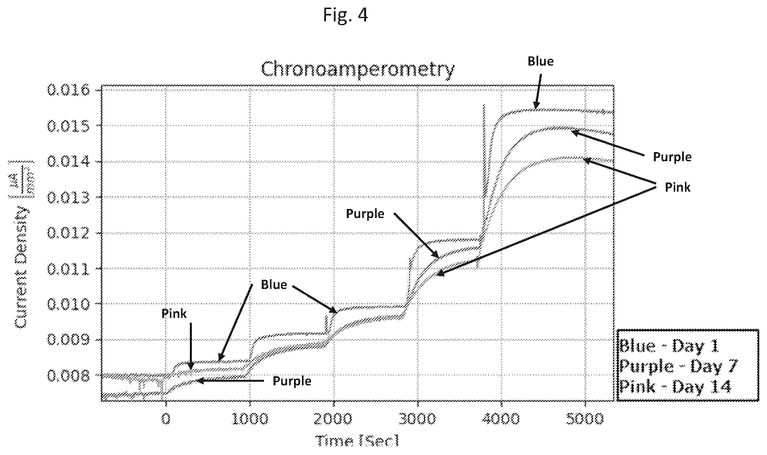

Example 2: same as example 1, except that the CDH enzyme (T4 from DirectSens, Vienna, Austria) has a Michaelis constant of about 80 mM for glucose (10 times higher than the Michaelis constant of the CDH enzyme used in Example 1). FIG. 4 shows chronoamperograms for this sensor. It is clearly seen that the response time increases over time, reaching an overly long value of 450 s after as little as 7 days.

While various inventive embodiments have been described and illustrated herein, those of ordinary skill in the art will readily envision a variety of other means, steps, components, and/or structures for performing the function of the embodiments (and elements thereof) disclosed herein, and/or obtaining the results and/or one or more of the advantages described herein, and each of such variations and/or modifications is deemed to be within the scope of the inventive embodiments described herein. More generally, those skilled in the art will readily appreciate that all parameters, amounts, dimensions, materials, steps, and configurations described herein are meant to be merely an example and that the actual parameters, amounts, dimensions, materials, steps, and configurations will depend upon the specific application or applications for which the inventive teachings is/are used. Those skilled in the art will recognize or be able to ascertain using no more than routine experimentation, many equivalents to the specific inventive embodiments described herein. It is therefore to be understood that the foregoing embodiments are presented by way of example only and that, within the scope of claims supported by the subject disclosure and equivalents thereto, and inventive embodiments may be practiced otherwise than as specifically described and claimed. Inventive embodiments of the present disclosure are directed to each individual feature, device, system, article, material, kit, step, function/functionality, and method described herein. In addition, any combination of two or more such features, devices, systems, articles, materials, kits, steps, functions/functionality, and methods, if such features, systems, articles, materials, kits, steps, functions/functionality, and methods are not mutually inconsistent, is included within the inventive scope of the present disclosure and considered embodiments.

Embodiments disclosed herein may also be combined with one or more features, components, materials, parameters, as well as complete systems, devices, and/or methods, to yield yet other embodiments and inventions. Moreover, some embodiments, may be distinguishable from the prior art by specifically lacking one and/or another feature disclosed in the particular prior art reference(s), i.e., claims to some embodiments may be distinguishable from the prior art by including one or more negative limitations.

Also, as noted, various inventive concepts may be embodied as one or more methods, of which one or more examples have been provided. The acts performed as part of the method(s) may be ordered in any suitable way. Accordingly, embodiments may be constructed in which acts are performed in an order different than illustrated, which may include performing some acts simultaneously, even though shown as sequential acts in illustrative embodiments.

Any and all references to publications or other documents, including but not limited to, patents, patent applications, articles, webpages, books, etc., presented anywhere in the present application, are herein incorporated by reference in their entirety. Moreover, all definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The terms “can” and “may” are used interchangeably in the present disclosure, and indicate that the referred to element, component, structure, function, functionality, objective, advantage, operation, step, process, apparatus, system, device, result, or clarification, has the ability to be used, included, or produced, or otherwise stand for the proposition indicated in the statement for which the term is used (or referred to) for a particular embodiment(s).

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A. and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements), etc.

In the claims, as well as in the specification above, all transitional phrases such as “comprising,” “including,” “carrying,” “having.” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A bio-electrochemical sensor for measuring the concentration of a substrate molecule comprising:
a working electrode;
at least one of a counter electrode and a reference electrode;
an enzyme layer including:
an oxygen insensitive enzyme capable of direct electron transfer and having a Michaelis constant of less than 10 mM with respect to the substrate molecule;
a polymer; and
a substrate limiting layer.

2. The sensor of claim 1, wherein the substrate comprises at least one of glucose, lactate and a ketone.

3. The sensor of claim 1, wherein the working electrode comprises at least one of gold and carbon.

4. The sensor of claim 1, wherein the enzyme layer is a genetically modified cellobiose dehydrogenase.

5. The sensor of claim 1, wherein the polymer is at least on of: polyethlyeneimine (PEI), branched polyethylenimine (BPEI), diethlyaminoethyl (DEAE) or polylysine.

6. The sensor of claim 1, wherein the polymer is an electrical conductor.

7. The sensor of claim 1, wherein the polymer includes poly(3,4-ethylenedioxythiophene) polystyrene sulfonte (PEDOT:PSS).

8. The sensor of claim 1, wherein the enzyme layer includes conductive particles that are made of at least one of carbon black and gold.

9. The sensor of claim 1 wherein the conductive particles have a size between 0.3 and 0.5 microns.

10. The sensor of claim 1 wherein the enzyme layer thickness exceeds 5 microns.

11. The sensor of claim 1, wherein the enzyme layer thickness is less than 5 microns.

12. The sensor of claim 1, wherein a voltage difference between the working electrode and the reference or counter/reference electrode is less than about 0.1V.

13. The sensor of claim 1 wherein the substrate limiting layer includes a polyurethane.

14. The sensor of claim 1 wherein the reduction factor afforded by the substrate limiting layer is less than 100.

15. (canceled)

16. (canceled)
